(19) United States
(12) Patent Application Publication
(10) Pub. No.: US 2023/0330088 A1
(43) Pub. Date: Oct. 19, 2023

(54) COMBINATION THERAPY FOR TREATING ABNORMAL CELL GROWTH

(71) Applicant: Verastem, Inc., Needham, MA (US)

(72) Inventors: Silvia COMA, Needham, MA (US); Jonathan A. PACHTER, Wayland, MA (US)

(21) Appl. No.: 18/005,012

(22) PCT Filed: Jul. 13, 2021
(86) PCT No.: PCT/US2021/041439

§ 371 Date: Jan. 10, 2023

Related U.S. Application Data
(60) Provisional application No. 63/051,320, filed on Jul. 13, 2020.

(51) Int. Cl. A61K 31/506; A61K 39/395; A61P 35/00; A61K 31/5377

(52) U.S. Cl. CPC A61K 31/506; A61K 39/3955; A61P 35/00; A61K 31/5377

(57) ABSTRACT

The present invention relates to methods, compositions, and oral dosage forms of a dual RAF/MEK inhibitor in combination with an anti-PD-1 antibody or an anti-PD-L1 antibody for treating abnormal cell growth (e.g., cancer).

### CROSS-REFERENCE TO RELATED APPLICATIONS

This application claims the benefit of U.S. Provisional Patent Application No. 63/051,320 filed on Jul. 13, 2020, the entire content of which is incorporated herein by reference.

### BACKGROUND

Components of the RAS/RAF/MEK/ERK (MAPK) signal transduction pathway represent opportunities for the treatment of abnormal cell growth, e.g., cancer. RAS and RAF are frequently mutated in human cancers. These mutants result in a constitutively active MAPK kinase cascade, leading to tumor cell proliferation, differentiation, survival, and migration. Selective inhibitors of certain components of the RAS/RAF/MEK/ERK signal transduction pathway, such as RAS, RAF, MEK and ERK, are useful in the treatment of abnormal cell growth, in particular cancer, in mammals.

Immune checkpoints refer to a plethora of inhibitory pathways that help maintain self-tolerance and modulate the duration and amplitude of physiological immune responses in peripheral tissues in order to minimize collateral tissue damage. Tumors co-opt certain immune checkpoint pathways as a mechanism of immune resistance, particularly against T-cells that are specific for tumor antigens. The development of checkpoint blocking antibodies, e.g., inhibitory receptors, that target or are directed against, for example, programmed death 1 receptor (PD-1), can facilitate the treatment of abnormal cell growth. PD-1 can function as negative regulators and have non-redundant roles in modulating immune responses. They are expressed on tumor-specific T-cells and can lead to compromised activation and suppressed effector functions e.g., proliferation, cytokine secretion, and tumor cell lysis. PD-1 is involved in modulating T-cell activity in e.g., peripheral tissues, e.g., via interaction with its ligands, i.e., PD-L1 and PD-L2. Blockers of the immune checkpoint pathway can enhance antitumor immunity and provide opportunities to treat abnormal cell growth and provide more effective treatment for subjects suffering from cancer.

Due to the severity and breadth of diseases and disorders associated with abnormal cell growth, e.g., cancer, there is a need for effective therapeutic means and methods for treatment. The compounds, compound combinations, compositions, and methods described herein are directed toward this end.

### SUMMARY

The present disclosure provides, in part, methods of treating abnormal cell growth (e.g., cancer) in a subject in need thereof. The methods comprise treating a subject in need thereof by administering a dual RAF/MEK inhibitor (e.g., VS-6766) or a pharmaceutically acceptable salt thereof in combination with an additional agent described herein (e.g., an anti-PD-1 antibody or an anti-PD-L1 antibody). The methods may further comprise administering to the subject a FAK inhibitor (e.g., defactinib) or a pharmaceutically acceptable salt thereof.

Thus, in an aspect, provided herein is a method of treating a cancer in a subject in need thereof, the method comprising administering to the subject a dual RAF/MEK inhibitor or a pharmaceutically acceptable salt thereof in combination with an anti-PD-1 antibody, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-L1 antibody, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-1 antibody and a FAK inhibitor, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-L1 antibody and a FAK inhibitor, thereby treating the subject.

In some embodiments, the dual RAF/MEK inhibitor is VS-6766 or a pharmaceutically acceptable salt thereof.

In some embodiments, the dual RAF/MEK inhibitor is dosed at least once a week. In some embodiments, the dual RAF/MEK inhibitor is dosed twice a week. In some embodiments, the dual RAF/MEK inhibitor is dosed twice a week cyclically for three weeks on and then one week off. In some embodiments, the cycle is repeated at least once.

In some embodiments, the dual RAF/MEK inhibitor is dosed at about 0.8 mg to about 10 mg per administration. In some embodiments, the dual RAF/MEK inhibitor is dosed at about 3.2 mg per administration. In some embodiments, the dual RAF/MEK inhibitor is dosed at about 4 mg per administration.

In some embodiments, the anti-PD-1 antibody is selected from the group consisting of balstilimab, camrelizumab, cemiplimab, dostarlimab, geptanolimab, nivolumab, pembrolizumab, penpulimab, pidilizumab, prolgolimab, retifanlimab, sasanlimab, serplulimab, serplulimab, sintilimab, spartalizumab, sulituzumab, tebotelimab, teripalimab, tislelizumab, toripalimab, toripalimab, zimberelimab, AMP-224 (MedImunne), AMP-514 (MedImunne), AT-16201 (AIMM Therapeutics BV), AVI-102 (AbVision Inc), BAT-1308 (Bio-Thera Solutions Ltd), BH-2950 (Beijing Hanmi Pharmaceutical Co Ltd), BSI-050K01 (Biosion Inc), CB-201 (Crescendo Biologics Ltd), CYTO-101 (Cytocom Inc), DB-004 (DotBio Pte Ltd), EX-105 (Excelmab Inc), EX-108 (Excelmab Inc), GNR-051 (Generium), HAB-21 (Suzhou Stainwei Biotech Inc), IBI-319 (Innovent Biologics Inc), IBI-321 (Innovent Biologics Inc), IKT-202 (Icell Kealex Therapeutics LLC), IMU-201 (Imugene Ltd), JS-201 (Shanghai Junshi Bioscience Co Ltd), LBL-006 (Leads Biolabs Inc), LBL-024 (Leads Biolabs Inc), LD-01 (Leidos Health Holdings LLC), LQ-005 (Shanghai Novamab Biopharmaceuticals Co Ltd), LQ-008 (Shanghai Novamab Biopharmaceuticals Co Ltd), MD-402 (MD Biosciences GmbH), OT-2 (OncoTrap Inc), PE-0105 (Shanghai Yunyi Health Technology Development Co Ltd), PF-07209960 (Pfizer Inc), PH-762 (Phio Pharmaceuticals Corp), REGN-PD-1/XX (Regeneron), R07121661 (Genentech), SAUG-1 (Juvenescence UK Ltd), SCT-IIOA (Sinocelltech), SG-001 (CSPC Pharmaceutical Group Ltd), SI-B003 (SystImmune), SL-279137 (Shattuck Labs), SSI-361 (Lyvgen Biopharma Ltd), STI-A1110 (Servier), STM-418 (Stcube Inc), Sym-021 (Symphogen A/S), TSR-075 (GlaxoSmithKline Plc), TY101 (Tayu Huaxia Biotech), Twist-PD-1 (Twist Bioscience), XmAb-TGFβR2 (Xencor), XmAb-YYCD28 (Xencor), XmAb20717 (Xencor), XmAb23104 (Xencor), YBL-006 (Y Biologics), YBL-019 (Y Biologics), and mDX-400 (Merck & Co Inc). In some embodiments, the anti-PD-1 antibody is selected from the group consisting of cemiplimab, nivolumab, pembrolizumab, pidilizumab, spartalizumab, camrelizumab, sintilimab, tislelizumab, toripalimab, dostarlimab, AMP-224, and AMP-514.

In some embodiments, the anti-PD-L1 antibody is selected from the group consisting of atezolizumab, avelumab, durvalumab, envafolimab, socazolimab, sugemalimab, ABM-101 (Abeome Corp), AP-505 (AP Biosciences Inc), APL-801 (Apollomics Inc), ATG-101 (Antengene Corp Ltd), AVA-027 (Avacta Life Sciences Ltd), AUNP12 (Aurigene), B-1961 (AP Biosciences Inc), BH-3120 (Hanmi Pharmaceuticals Co Ltd), BMS-986189 (Bristol Myers Squibb), BPI-9220 (Beta Pharma Inc), BPI-9320 (Beta Pharma Inc), CA-170 (Curis Inc), CCX-559 (ChemoCentryx Inc), CK-301 (cosibelimab), CS-17938 (Shenzhen Chipscreen Biosciences Co Ltd), CTX-8371 (Compass Therapeutics Inc), CYTCDR-2 (CytImmune Sciences Inc), DB-003 (DotBio Pte Ltd), DF-002 (Suzhou Dingfu Target Biotechnology Co Ltd), DPDL-1E (Shanghai Hycharm Inc), DR-30207 (Zhejiang Doer Biologics Corp), DSP-105 (KAHR medical Ltd), DSP-502 (KAHR medical Ltd), EI-011 (Elixiron Immunotherapeutics Inc), EI-014 (Elixiron Immunotherapeutics Inc), EMB-08 (EpimAb Biotherapeutics Inc), ENN-101 (Ennovabio), ENN-102 (Ennovabio), GB-7003 (Shanghai GeneChem Co Ltd), Gensci-047 (GeneScience Pharmaceuticals Co Ltd), HB-0025 (Huabo Biopharm (Shanghai) Co Ltd), HB-0028 (Huabo Biopharm (Shanghai) Co Ltd), HB-0036 (Huabo Biopharm (Shanghai) Co Ltd), HBM-7015 (Harbour BioMed (Guangzhou) Co Ltd), IBI-327 (Innovent Biologics Inc), IGM-7354 (IGM Biosciences Inc), IKT-201 (Icell Kealex Therapeutics LLC), IMC-2101 (ImmuneOncia Therapeutics LLC), IMC-2102 (ImmuneOncia Therapeutics LLC), IMGS-002 (Immunogenesis Inc), IMM-2510 (ImmuneOnco Biopharmaceuticals (Shanghai) Co Ltd), INBRX-105 (Elpiscience Biopharmaceutical Ltd), JBI-426 (Jubilant Therapeutics Inc), JNB-809 (JN Biosciences LLC), JNB-813 (JN Biosciences LLC), KN-052 (Alphamab Oncology), KY-1043 (Kymab Ltd), LP-008 (Lepu Biopharma Co Ltd), LQ-002 (Shanghai Novamab Biopharmaceuticals Co Ltd), LQ-004 (Shanghai Novamab Biopharmaceuticals Co Ltd), LVGN-1673 (Lyvgen Biopharma Ltd), LY-3434172 (Eli Lilly and Co), LYN-102 (LynkCell Inc), MCLA-145 (Merus NV), MEDI-7526 (AstraZeneca Plc), PH-790 (Phio Pharmaceuticals Corp), PM-1003 (Biotheus Inc), PRS-344 (Pieris Pharmaceuticals Inc), Q-1802 (QureBio), QL-301 (QLSF Biotherapeutics Inc), QLS31901 (Qilu Pharmaceutical), RC98 (RemeGen), SHR-1316 (Jiangsu Hengrui Medicine Co Ltd), SHR-1701 (Jiangsu Hengrui Medicine Co Ltd), SIM-236 (Jiangsu Simcere Pharmaceutical Co Ltd), SL-279252 (Shattuck Labs Inc), SL-279258 (Shattuck Labs Inc), SLSP-03 (Salspera LLC), SNA-02 (Oneness Biotech Co Ltd), STT-01 (Stcube Inc), TI-1007 (Timmune Biotech), TJ-L1C4 (I-Mab Biopharma), TJ-L1D5 (I-Mab Biopharma), TJ-L1H3 (I-Mab Biopharma), TJ-L117 (I-Mab Biopharma), TJL-14B (I-Mab Biopharma), TS1905 (Luye Pharma Group), TST-005 (Transcenta Holding Ltd), TTXsiPDL-1 (Transcode Therapeutics Inc), TXB-4BC3 (Ossianix Inc), VXM-10 (Vaximm AG), YBL-007 (Y-Biologics Inc), YBL-008 (Y-Biologics Inc), YBL-009 (Y-Biologics Inc), YBL-013 (Y-Biologics Inc), YBL-016 (Y-Biologics Inc), YBL-020 (Y-Biologics Inc). In some embodiments, the anti-PD-L1 antibody is selected from the group consisting of avelumab, durvalumab, atezolizumab, KN035, CK-301, AUNP12, CA-170, and BMS-986189.

In some embodiments, the FAK inhibitor is defactinib.

In some embodiments, the cancer is a cancer characterized as having a RAS mutation. In some embodiments, the cancer is a cancer characterized as having a RAF mutation. In some embodiments, the cancer is a cancer characterized as having a KRAS, NRAS, HRAS, and/or BRAF mutation.

In some embodiments, the cancer is lung adenocarcinoma, colorectal cancer, uveal melanoma, ovarian cancer, uterine endometrioid carcinoma, bladder urothelial carcinoma, breast invasive lobular carcinoma, cervical squamous cell carcinoma, cutaneous melanoma, endocervical adenocarcinoma, hepatocellular carcinoma, pancreatic adenocarcinoma, biphasic type pleural mesothelioma, renal clear cell carcinoma, renal clear cell carcinoma, stomach adenocarcinoma, tubular stomach adenocarcinoma, uterine carcinosarcoma, or uterine malignant mixed Mullerian tumor.

Other objects and advantages will become apparent to those skilled in the art from a consideration of the ensuing Detailed Description, Examples, and Claims.

### BRIEF DESCRIPTION OF THE DRAWINGS

FIG. 1 shows that VS-6766 upregulates markers associated with antigen presentation.

FIG. 2A shows exemplary tumor volume changes in CT26 xenografts after treatment with VS-6766 and/or anti-PD-1 antibody.

FIG. 2B shows exemplary tumor volume changes in CT26 mice treated with VS-6766 and/or anti-PD-1 antibody.

FIG. 2C shows an exemplary Kaplan-Meier survival curve of CT26 mice treated with VS-6766 and/or anti-PD-1 antibody.

FIG. 3A shows immune memory in an exemplary CT26 colorectal cancer model.

FIG. 3B shows exemplary effector memory in CD4 or CD8 cells.

FIG. 4A shows exemplary antitumor effects of VS-6766+anti-PD-1 in combination with a FAK inhibitor.

FIG. 4B shows exemplary antitumor effects of VS-6766+FAK inhibitor in combination with an anti-PD-1.

### DETAILED DESCRIPTION

As generally described herein, the present disclosure provides methods and combinations of compounds useful for treating abnormal cell growth (e.g., cancer) in a subject in need thereof.

### Definitions

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, “prophylactic treatment” contemplates an action that occurs before a subject begins to suffer from the specified disease, disorder or condition.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

The term, “oral dosage form,” as used herein, refers to a composition or medium used to administer an agent to a subject. Typically, an oral dosage form is administered via the mouth, however, “oral dosage form” is intended to cover any substance which is administered to a subject and is absorbed across a membrane, e.g., a mucosal membrane, of the gastrointestinal tract, including, e.g., the mouth, esophagus, stomach, small intestine, large intestine, and colon. For example, “oral dosage form” covers a solution which is administered through a feeding tube into the stomach.

A “KRAS mutation” is a mutation of the KRAS gene (i.e., a nucleic acid mutation) or Kras protein (i.e., an amino acid mutation) that results in aberrant Kras protein function associated with increased and/or constitutive activity by favoring the active GTP-bound state of the Kras protein. The mutation may be at conserved sites that favor GTP binding and constitutively active Kras protein. In some instances, the mutation is at one or more of codons 12, 13, and 16 of the KRAS gene. For example, a KRAS mutation may be at codon 12 of the KRAS gene, for instance, as a single point substitution mutation at codon 12 (i.e., KRAS G12X mutation) (e.g., a KRAS G12V mutation arises from a single nucleotide change (c.35G>T) and results in an amino acid substitution of the glycine (G) at position 12 by a valine (V)). Exemplary KRAS G12X mutations include, but are not limited to, KRAS G12V, KRAS G12D, KRAS G12A, KRAS G12R, KRAS G12S, or KRAS G12C.

### Methods of Treatment

Combinations of compounds described herein (e.g., a dual RAF/MEK inhibitor in combination with an anti-PD-1 antibody or an anti-PD-L1 antibody) and pharmaceutical compositions thereof are generally useful in methods of treating abnormal cell growth such as cancer.

Thus, in an aspect, provided herein is a method of treating a cancer in a subject in need thereof, the method comprising administering to the subject a dual RAF/MEK inhibitor or a pharmaceutically acceptable salt thereof in combination with an anti-PD-1 antibody, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-L1 antibody, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-1 antibody and a FAK inhibitor, thereby treating the subject.

In another aspect, provided herein is a method of treating a cancer in a subject in need thereof, the method comprising administering to the subject a dual RAF/MEK inhibitor or a pharmaceutically acceptable salt thereof in combination with an anti-PD-L1 antibody and a FAK inhibitor, thereby treating the subject.

In some embodiments, the methods described herein induces immune memory.

### Dual RAF/MEK Inhibitors

An exemplary dual RAF/MEK inhibitor described herein is VS-6766 (also referred to as CK127, CH5126766, or RO5126766) having the following structure:

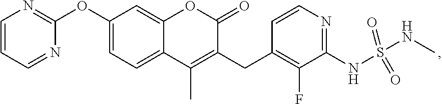 [SMILES: CNS(=O)(=O)Nc1nccc(Cc2c(C)c3ccc(Oc4ncccn4)cc3oc2=O)c1F]

or a pharmaceutically acceptable salt thereof.

In some embodiments, the pharmaceutically acceptable salt of VS-6766 is a potassium salt of VS-6766.

In some embodiments, the dual RAF/MEK inhibitor is dosed at least once a week (e.g., once a week, twice a week, three times a week, four times a week, five times a week, or six times a week). In some embodiments, the dual RAF/MEK inhibitor is dosed once a week. In some embodiments, the dual RAF/MEK inhibitor is dosed twice a week. In some embodiments, the dual RAF/MEK inhibitor is dosed three times a week.

In some embodiments, the dual RAF/MEK inhibitor is dosed at about 0.1 mg to about 100 mg, e.g., about 0.1 mg to about 50 mg, about 0.1 mg to about 10 mg, about 0.1 mg to about 5 mg, about 0.1 mg to about 4 mg, about 0.1 mg to about 3 mg, about 0.1 mg to about 2 mg, about 0.1 mg to about 1 mg, about 1 mg to about 10 mg, about 1 mg to about 20 mg, about 1 mg to about 40 mg, about 1 mg to about 60 mg, about 1 mg to about 80 mg, about 1 mg to about 100 mg, about 10 mg to about 100 mg, about 20 mg to about 100 mg, about 40 mg to about 100 mg, about 60 mg to about 100 mg, or about 80 mg to about 100 mg. In some embodiments, the dual RAF/MEK inhibitor is dosed at about 0.5 mg to about 10 mg per administration. In some embodiments, dual RAF/MEK inhibitor is dosed at about 0.8 mg to about 10 mg per administration. In some embodiments, the dual RAF/MEK inhibitor is dosed at about 0.1 mg, 0.2 mg, 0.5 mg, 1 mg, 1.5 mg, 3 mg, 4 mg, 5 mg, 10 mg, 15 mg, 20 mg, 25 mg, 30 mg, 35 mg, 40 mg, 45 mg, 50 mg, 65 mg, 70 mg, 75 mg, 80 mg, 85 mg, 90 mg, 95 mg, or 100 mg per administration. In some embodiments, dual RAF/MEK inhibitor is dosed at about 4 mg per administration. In some embodiments, the dual RAF/MEK inhibitor is dosed at about 3.2 mg per administration. In some embodiments, the dual RAF/MEK inhibitor is administered orally.

In some embodiments, the dual RAF/MEK inhibitor is dosed cyclically for three weeks on and then one week off. In some embodiments, the dual RAF/MEK inhibitor is dosed twice a week. In some embodiments, the dual RAF/MEK inhibitor is dosed three times a week. In some embodiments, the dual RAF/MEK inhibitor is dosed at about 0.8 mg to about 10 mg (e.g., about 4 mg or about 3.2 mg) per administration.

In some embodiments, the dual RAF/MEK inhibitor is dosed twice a week cyclically for three weeks on and then one week off at a dose of about 0.8 mg to about 10 mg per administration (e.g., about 4 mg or about 3.2 mg per administration). In some embodiments, the cycle (e.g., three weeks on and one week off) is repeated at least once.

In some embodiments, the dual RAF/MEK inhibitor is dosed three times a week cyclically for three weeks on and then one week off at a dose of about 0.8 mg to about 10 mg per administration (e.g., about 4 mg or about 3.2 mg per administration). In some embodiments, the cycle (e.g., three weeks on and one week off) is repeated at least once.

In alternative embodiments, the dual RAF/MEK inhibitor is dosed continuously (i.e., without the three weeks on and then one week off cycle). In some embodiments, the dual RAF/MEK inhibitor is dosed twice a week. In some embodiments, the dual RAF/MEK inhibitor is dosed three times a week. In some embodiments, the dual RAF/MEK inhibitor is dosed at about 0.8 mg to about 10 mg (e.g., about 4 mg or about 3.2 mg) per administration. In some embodiments, the dual RAF/MEK inhibitor is dosed for at least four weeks. In some embodiments, the dual RAF/MEK inhibitor is dosed for four weeks.

In some embodiments, the dual RAF/MEK inhibitor is administered to the patient twice a week at a dose of about 0.8 mg to about 10 mg per administration (e.g., about 4 mg or about 3.2 mg per administration) then dosed cyclically for three weeks on and then one week off, wherein the cycle is repeated at least once. In some embodiments, the dual RAF/MEK inhibitor is dosed twice a week cyclically for three weeks on and then one week off at a dose of about 0.8 mg to about 10 mg per administration (e.g., about 4 mg or about 3.2 mg per administration).

In some embodiments, the dual RAF/MEK inhibitor is administered to the patient three times a week at a dose of about 0.8 mg to about 10 mg per administration (e.g., about 4 mg or about 3.2 mg per administration) then dosed cyclically for three weeks on and then one week off, wherein the cycle is repeated at least once. In some embodiments, the dual RAF/MEK inhibitor is dosed three times a week cyclically for three weeks on and then one week off at a dose of about 0.8 mg to about 10 mg per administration (e.g., about 4 mg or about 3.2 mg per administration).

### Anti-PD-1 Antibodies/Anti-PD-L1 Antibodies

Antibody therapies are antibody proteins produced by the immune system and that bind to a target antigen on the surface of a cell. Antibodies are typically encoded by an immunoglobulin gene or genes, or fragments thereof. In normal physiology antibodies are used by the immune system to fight pathogens. Each antibody is specific to one or a few proteins, and those that bind to cancer antigens are used, e.g., for the treatment of cancer. Antibodies are capable of specifically binding an antigen or epitope. (Fundamental Immunology, 3^{rd} P Edition, W. e., Paul, ed., Raven Press, N.Y. (1993). Specific binding occurs to the corresponding antigen or epitope even in the presence of a heterogeneous population of proteins and other biologies. Specific binding of an antibody indicates that it binds to its target antigen or epitope with an affinity that is substantially greater than binding to irrelevant antigens. The relative difference in affinity is often at least 25% greater, more often at least 50% greater, most often at least 100% greater. The relative difference can be at least 2-fold, at least 5-fold, at least 10-fold, at least 25-fold, at least 50-fold, at least 100-fold, or at least 1000-fold, for example.

Exemplary types of antibodies include without limitation human, humanized, chimeric, monoclonal, polyclonal, single chain, antibody binding fragments, and diabodies. Once bound to a cancer antigen, antibodies can induce antibody-dependent cell-mediated cytotoxicity, activate the complement system, prevent a receptor interacting with its ligand or deliver a payload of chemotherapy or radiation, all of which can lead to cell death.

In some embodiments, the anti-PD-1 antibody is selected from the group consisting of balstilimab, camrelizumab, cemiplimab, dostarlimab, geptanolimab, nivolumab, pembrolizumab, penpulimab, pidilizumab, prolgolimab, retifanlimab, sasanlimab, serplulimab, serplulimab, sintilimab, spartalizumab, sulituzumab, tebotelimab, teripalimab, tislelizumab, toripalimab, toripalimab, zimberelimab, AMP-224 (MedImunne), AMP-514 (MedImunne), AT-16201 (AIMM Therapeutics BV), AVI-102 (AbVision Inc), BAT-1308 (Bio-Thera Solutions Ltd), BH-2950 (Beijing Hanmi Pharmaceutical Co Ltd), BSI-050K01 (Biosion Inc), CB-201 (Crescendo Biologics Ltd), CYTO-101 (Cytocom Inc), DB-004 (DotBio Pte Ltd), EX-105 (Excelmab Inc), EX-108 (Excelmab Inc), GNR-051 (Generium), HAB-21 (Suzhou Stainwei Biotech Inc), IBI-319 (Innovent Biologics Inc), IBI-321 (Innovent Biologics Inc), IKT-202 (Icell Kealex Therapeutics LLC), IMU-201 (Imugene Ltd), JS-201 (Shanghai Junshi Bioscience Co Ltd), LBL-006 (Leads Biolabs Inc), LBL-024 (Leads Biolabs Inc), LD-01 (Leidos Health Holdings LLC), LQ-005 (Shanghai Novamab Biopharmaceuticals Co Ltd), LQ-008 (Shanghai Novamab Biopharmaceuticals Co Ltd), MD-402 (MD Biosciences GmbH), OT-2 (OncoTrap Inc), PE-0105 (Shanghai Yunyi Health Technology Development Co Ltd), PF-07209960 (Pfizer Inc), PH-762 (Phio Pharmaceuticals Corp), REGN-PD-1/XX (Regeneron), R07121661 (Genentech), SAUG-1 (Juvenescence UK Ltd), SCT-IIOA (Sinocelltech), SG-001 (CSPC Pharmaceutical Group Ltd), SI-B003 (SystImmune), SL-279137 (Shattuck Labs), SSI-361 (Lyvgen Biopharma Ltd), STI-A1110 (Servier), STM-418 (Stcube Inc), Sym-021 (Symphogen A/S), TSR-075 (GlaxoSmithKline Plc), TY101 (Tayu Huaxia Biotech), Twist-PD-1 (Twist Bioscience), XmAb-TGFβR2 (Xencor), XmAb-YYCD28 (Xencor), XmAb20717 (Xencor), XmAb23104 (Xencor), YBL-006 (Y Biologics), YBL-019 (Y Biologics), and mDX-400 (Merck & Co Inc). In some embodiments, the anti-PD-1 antibody is selected from the group consisting of cemiplimab, nivolumab, pembrolizumab, pidilizumab, spartalizumab, camrelizumab, sintilimab, tislelizumab, toripalimab, dostarlimab, AMP-224 (NCI), and AMP-514. In some embodiments, the anti-PD-1 antibody is nivolumab. In some embodiments, the anti-PD-1 antibody is pembrolizumab.

In some embodiments, the anti-PD-1 antibody is dosed at least once a week. In some embodiments, the anti-PD-1 antibody is dosed once a week. In some embodiments, the anti-PD-1 antibody is dosed twice a week. In other embodiments, the anti-PD-1 antibody is dosed every 2 weeks. In other embodiments, the anti-PD-1 antibody is dosed every 3 weeks. In other embodiments, the anti-PD-1 antibody is dosed every 4 weeks. In other embodiments, the anti-PD-1 antibody is dosed every 5 weeks. In other embodiments, the anti-PD-1 antibody is dosed every 6 weeks.

In some embodiments, the anti-PD-1 antibody is dosed at about 100 mg to about 2000 mg, about 100 mg to about 1500 mg, about 100 mg to about 1000 mg, about 100 mg to about 800 mg, about 100 mg to about 500 mg, about 200 mg to about 500 mg, (e.g., about 200 mg, 240 mg, or about 480 mg) per administration.

In some embodiments, the anti-PD-1 antibody is administered parenterally (e.g., intravenous infusion).

In some embodiments, the anti-PD-L1 antibody is selected from the group consisting of atezolizumab, avelumab, durvalumab, envafolimab, socazolimab, sugemalimab, ABM-101 (Abeome Corp), AP-505 (AP Biosciences Inc), APL-801 (Apollomics Inc), ATG-101 (Antengene Corp Ltd), AVA-027 (Avacta Life Sciences Ltd), AUNP12 (Aurigene), B-1961 (AP Biosciences Inc), BH-3120 (Hanmi Pharmaceuticals Co Ltd), BMS-986189 (Bristol Myers Squibb), BPI-9220 (Beta Pharma Inc), BPI-9320 (Beta Pharma Inc), CA-170 (Curis Inc), CCX-559 (ChemoCentryx Inc), CK-301 (cosibelimab), CS-17938 (Shenzhen Chipscreen Biosciences Co Ltd), CTX-8371 (Compass Therapeutics Inc), CYTCDR-2 (CytImmune Sciences Inc), DB-003 (DotBio Pte Ltd), DF-002 (Suzhou Dingfu Target Biotechnology Co Ltd), DPDL-1E (Shanghai Hycharm Inc), DR-30207 (Zhejiang Doer Biologics Corp), DSP-105 (KAHR medical Ltd), DSP-502 (KAHR medical Ltd), EI-011 (Elixiron Immunotherapeutics Inc), EI-014 (Elixiron Immunotherapeutics Inc), EMB-08 (EpimAb Biotherapeutics Inc), ENN-101 (Ennovabio), ENN-102 (Ennovabio), GB-7003 (Shanghai GeneChem Co Ltd), Gensci-047 (GeneScience Pharmaceuticals Co Ltd), HB-0025 (Huabo Biopharm (Shanghai) Co Ltd), HB-0028 (Huabo Biopharm (Shanghai) Co Ltd), HB-0036 (Huabo Biopharm (Shanghai) Co Ltd), HBM-7015 (Harbour BioMed (Guangzhou) Co Ltd), IBI-327 (Innovent Biologics Inc), IGM-7354 (IGM Biosciences Inc), IKT-201 (Icell Kealex Therapeutics LLC), IMC-2101 (ImmuneOncia Therapeutics LLC), IMC-2102 (ImmuneOncia Therapeutics LLC), IMGS-002 (Immunogenesis Inc), IMM-2510 (ImmuneOnco Biopharmaceuticals (Shanghai) Co Ltd), INBRX-105 (Elpiscience Biopharmaceutical Ltd), JBI-426 (Jubilant Therapeutics Inc), JNB-809 (JN Biosciences LLC), JNB-813 (JN Biosciences LLC), KN-052 (Alphamab Oncology), KY-1043 (Kymab Ltd), LP-008 (Lepu Biopharma Co Ltd), LQ-002 (Shanghai Novamab Biopharmaceuticals Co Ltd), LQ-004 (Shanghai Novamab Biopharmaceuticals Co Ltd), LVGN-1673 (Lyvgen Biopharma Ltd), LY-3434172 (Eli Lilly and Co), LYN-102 (LynkCell Inc), MCLA-145 (Merus NV), MEDI-7526 (AstraZeneca Plc), PH-790 (Phio Pharmaceuticals Corp), PM-1003 (Biotheus Inc), PRS-344 (Pieris Pharmaceuticals Inc), Q-1802 (QureBio), QL-301 (QLSF Biotherapeutics Inc), QLS31901 (Qilu Pharmaceutical), RC98 (RemeGen), SHR-1316 (Jiangsu Hengrui Medicine Co Ltd), SHR-1701 (Jiangsu Hengrui Medicine Co Ltd), SIM-236 (Jiangsu Simcere Pharmaceutical Co Ltd), SL-279252 (Shattuck Labs Inc), SL-279258 (Shattuck Labs Inc), SLSP-03 (Salspera LLC), SNA-02 (Oneness Biotech Co Ltd), STT-01 (Stcube Inc), TI-1007 (Timmune Biotech), TJ-L1C4 (I-Mab Biopharma), TJ-L1D5 (I-Mab Biopharma), TJ-L1H3 (I-Mab Biopharma), TJ-L117 (I-Mab Biopharma), TJL-14B (I-Mab Biopharma), TS1905 (Luye Pharma Group), TST-005 (Transcenta Holding Ltd), TTXsiPDL-1 (Transcode Therapeutics Inc), TXB-4BC3 (Ossianix Inc), VXM-10 (Vaximm AG), YBL-007 (Y-Biologics Inc), YBL-008 (Y-Biologics Inc), YBL-009 (Y-Biologics Inc), YBL-013 (Y-Biologics Inc), YBL-016 (Y-Biologics Inc), and YBL-020 (Y-Biologics Inc). In some embodiments, the anti-PD-L1 antibody is selected from the group consisting of avelumab, durvalumab, atezolizumab, KN035, CK-301, AUNP12, CA-170, and BMS-986189.

In some embodiments, the anti-PD-L1 antibody is dosed at least once a week. In some embodiments, the anti-PD-L1 antibody is dosed once a week. In some embodiments, the anti-PD-L1 antibody is dosed twice a week. In other embodiments, the anti-PD-L1 antibody is dosed every 2 weeks. In other embodiments, the anti-PD-L1 antibody is dosed every 3 weeks. In other embodiments, the anti-PD-L1 antibody is dosed every 4 weeks. In other embodiments, the anti-PD-L1 antibody is dosed every 5 weeks. In other embodiments, the anti-PD-L1 antibody is dosed every 6 weeks.

In some embodiments, the anti-PD-L1 antibody is dosed at about 100 mg to about 2000 mg, about 100 mg to about 1500 mg, about 100 mg to about 1000 mg, about 100 mg to about 800 mg, about 100 mg to about 500 mg, about 200 mg to about 500 mg, about 500 mg to about 1500 mg, about 500 mg to about 1200 mg, about 800 mg to about 1200 mg, about 800 mg to about 1500 mg, per administration. For example, the anti-PD-L1 antibody may be dosed at about 400 mg, about 800 mg, or about 1200 mg per administration.

In some embodiments, the anti-PD-L1 antibody is administered parenterally (e.g., intravenous infusion).

### FAK Inhibitors

Potent inhibitors of the FAK protein tyrosine kinases may be adapted to therapeutic use as antiproliferative agents (e.g., anticancer), antitumor (e.g., effective against solid tumors), antiangiogenesis (e.g., stop or prevent proliferation of blood vessels) in mammals, particularly in humans. In some embodiments, the methods described herein further contemplate administering to the subject a FAK inhibitor described herein. The FAK inhibitors may be useful in the prevention and treatment of non-hematologic malignancies, a variety of human hyperproliferative disorders such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions such as benign hyperplasia of the skin (e.g., psoriasis) and benign hyperplasia of the prostate (e.g., BPH), and in the prevention and treatment of disorders such as mesothelioma. In some embodiments, the compounds described herein, e.g., FAK inhibitors, inhibit protein tyrosine kinase 2 (PYK2).

An exemplary FAK inhibitor includes, but is not limited to, defactinib having the following structure:

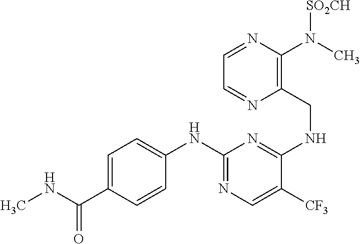

or a pharmaceutically acceptable salt thereof. Defactinib is also known as VS-6063 (e.g., VS-6063 free base) or PF-04554878. VS-6063 and related compounds are also disclosed in, for example, U.S. Pat. No. 7,928,109, the content of which is incorporated herein by reference. In some embodiments, VS-6063 can form a pharmaceutically acceptable salt (e.g., VS-6063 hydrochloride).

In some embodiments, the FAK inhibitor is VS-4718, having the following structure:

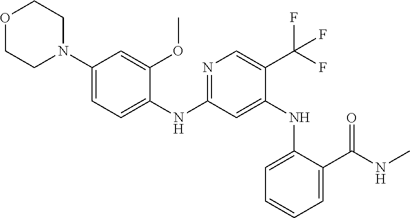 [SMILES: CNC(=O)c1ccccc1Nc1cc(Nc2ccc(N3CCOCC3)cc2OC)ncc1C(F)(F)F]

or a pharmaceutically acceptable salt thereof.

In some embodiments, the FAK inhibitor is TAE226, having the following structure:

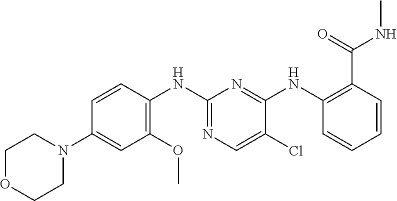 [SMILES: CNC(=O)c1ccccc1Nc1nc(Nc2ccc(N3CCOCC3)cc2OC)ncc1Cl]

or a pharmaceutically acceptable salt thereof.

In some embodiments, the FAK inhibitor is GSK2256098, having the following structure:

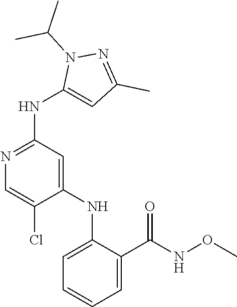 [SMILES: CONC(=O)c1ccccc1Nc1cc(Nc2cc(C)nn2C(C)C)ncc1Cl]

or a pharmaceutically acceptable salt thereof.

In some embodiments, the FAK inhibitor is PF-03814735, having the following structure:

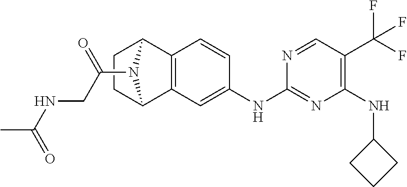 [SMILES: CC(=O)NCC(=O)N1[C@@H]2CC[C@H]1c1ccc(Nc3ncc(C(F)(F)F)c(NC4CCC4)n3)cc12]

or a pharmaceutically acceptable salt thereof.

In some embodiments, the FAK inhibitor is BI-4464, having the following structure:

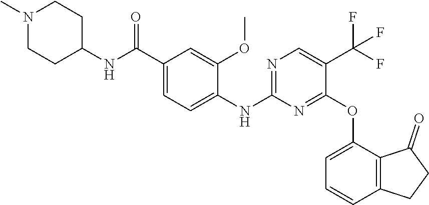 [SMILES: COc1cc(C(=O)NC2CCN(C)CC2)ccc1Nc1ncc(C(F)(F)F)c(Oc2cccc3c2C(=O)CC3)n1]

or a pharmaceutically acceptable salt thereof.

In some embodiments, the FAK inhibitor is BI-853520 (IN10018; Boehringer Ingelheim). In some other embodiments, the FAK inhibitor is APG-2449 (Ascentage Pharma Group).

In some embodiments, the FAK inhibitor is selected from the group consisting of defactinib, TAE226, BI-853520, GSK2256098, PF-03814735, BI-4464, VS-4718, and APG-2449, or a pharmaceutically acceptable salt thereof. For example, the FAK inhibitor is defactinib or a pharmaceutically acceptable salt thereof.

In some embodiments, the FAK inhibitor (e.g., defactinib) is dosed at least once daily. For example, in some embodiments, the FAK inhibitor (e.g., defactinib) is dosed twice daily. In some embodiments, the FAK inhibitor (e.g., defactinib) is dosed once daily.

In some embodiments, the FAK inhibitor (e.g., defactinib) is dosed at about 100 mg to about 1000 mg, e.g., about 100 mg to about 800 mg, about 100 mg to about 600 mg, about 100 mg to about 400 mg, about 100 mg to about 200 mg, about 200 mg to about 1000 mg, about 400 mg to about 1000 mg, about 600 mg to about 1000 mg, about 800 mg to about 1000 mg, about 200 mg to about 800 mg, about 200 mg to about 600 mg, about 200 mg to about 400 mg, about 400 mg to about 800 mg, or about 400 mg to about 600 mg per administration. In some embodiments, the FAK inhibitor (e.g., defactinib) is dosed at about 200 mg to about 400 mg per administration. In some embodiments, the FAK inhibitor (e.g., defactinib) is dosed at about 100 mg per administration. In some embodiments, the FAK inhibitor (e.g., defactinib) is dosed at about 200 mg per administration. In some embodiments, the FAK inhibitor (e.g., defactinib) is dosed at about 300 mg per administration. In some embodiments, the FAK inhibitor (e.g., defactinib) is dosed at about 400 mg per administration. In some embodiments, the FAK inhibitor (e.g., defactinib) is dosed at about 500 mg per administration. In some embodiments, the FAK inhibitor (e.g., defactinib) is dosed at about 600 mg per administration. In some embodiments, the FAK inhibitor (e.g., defactinib) is administered orally.

### Diseases and Disorders

### Abnormal Cell Growth

Abnormal cell growth, as used herein and unless otherwise indicated, refers to cell growth that is independent of normal regulatory mechanisms (e.g., loss of contact inhibition). This includes the abnormal growth of: (1) tumor cells (tumors) that proliferate, for example, by expressing a mutated tyrosine kinase or overexpression of a receptor tyrosine kinase; (2) benign and malignant cells of other proliferative diseases, for example, in which aberrant tyrosine kinase activation occurs; (3) any tumors that proliferate, for example, by receptor tyrosine kinases; (4) any tumors mat proliferate, for example, by aberrant serine/threonine kinase activation; and (5) benign and malignant cells of other proliferative diseases, for example, in which aberrant serine/threonine kinase activation occurs. Abnormal cell growth can refer to cell growth in epithelial (e.g., carcinomas, adenocarcinomas): mesenchymal (e.g., sarcomas (e.g. leiomyosarcoma. Ewing's sarcoma)); hematopoetic (e.g., lymphomas, leukemias, myelodysplasias (e.g., pre-malignant)); or other (e.g., melanoma, mesothelioma, and other tumors of unknown origin) cell.

### Neoplastic Disorders

Abnormal cell growth can refer to a neoplastic disorder. A “neoplastic disorder” is a disease or disorder characterized by cells that have the capacity for autonomous growth or replication, e.g., an abnormal state or condition characterized by proliferative cell growth. An abnormal mass of tissue as a result of abnormal cell growth or division, or a “neoplasm,” can be benign, pre-malignant (carcinoma in situ) or malignant (cancer).

Exemplary neoplastic disorders include: carcinoma, sarcoma, metastatic disorders (e.g., tumors arising from prostate, colon, lung, breast and liver origin), hematopoietic neoplastic disorders, e.g., leukemias, metastatic tumors. Treatment with the compound may be in an amount effective to ameliorate at least one symptom of the neoplastic disorder, e.g., reduced cell proliferation, reduced tumor mass, etc.

### Cancer

The inventive methods of the present invention may be useful in the prevention and treatment of cancer, including for example, solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon), and genitourinary (e.g., renal, urothelial, or testicular tumors) tracts, pharynx, prostate, and ovary. Exemplary adenocarcinomas include colorectal cancers, renal-cell carcinoma, liver cancer (e.g. Hepatocellular carcinoma), non-small cell carcinoma of the lung, pancreatic (e.g., metastatic pancreatic adenocarcinoma) and cancer of the small intestine.

The cancer can include mesothelioma; neurofibromatosis; e.g., neurofibromatosis type 2, neurofibromatosis type 1; renal cancer; lung cancer, non small cell lung cancer; liver cancer; thyroid cancer; ovarian; breast cancer; a nervous system tumor; schwannoma; meningioma; schwannomatosis; neuroma acoustic; adenoid cystic carcinoma; ependymoma; ependymal tumors, or any other tumor which exhibits decreased merlin expression and/or mutation, and/or deletion and/or promotor hypermethylation of the NF-2 gene. In some embodiments, the cancer is renal cancer.

The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells (e.g., a tumor enriched with cells that have undergone an epithelial-to-mesenchymal transition or a metastatic tumor).

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor.

The cancer can also include for example, but is not limited to, epithelial cancers, breast, lung, pancreatic, colorectal (e.g., metastatic colorectal, e.g., metastatic KRAS mutated), prostate, head and neck, melanoma (e.g., NRAS mutated locally advanced or metastatic malignant cutaneous melanoma), acute myelogenous leukemia, and glioblastoma. Exemplary breast cancers include triple negative breast cancer, basal-like breast cancer, claudin-low breast cancer, invasive, inflammatory, metaplastic, and advanced HER-2 positive or ER-positive cancers resistant to therapy.

In some embodiments, the cancer includes a cancer characterized as having a RAS mutation. The cancer can also include a cancer characterized as having a KRAS mutation. The cancer can also include a cancer characterized as having a NRAS mutation. The cancer can also include a cancer characterized as having a HRAS mutation.

In some embodiments, the cancer can also include a cancer characterized as having a RAF mutation. In some embodiments, the cancer can also include a cancer characterized as having a BRAF mutation.

The cancer can also include lung adenocarcinoma, colorectal cancer (CRC), uveal melanoma, ovarian cancer, uterine endometrioid carcinoma, bladder urothelial carcinoma, breast invasive lobular carcinoma, cervical squamous cell carcinoma, cutaneous melanoma, endocervical adenocarcinoma, hepatocellular carcinoma, pancreatic adenocarcinoma, biphasic type pleural mesothelioma, renal clear cell carcinoma, renal clear cell carcinoma, stomach adenocarcinoma, tubular stomach adenocarcinoma, uterine carcinosarcoma, or uterine malignant mixed Mullerian tumor.

In some embodiments, the cancer is unresectable or metastatic melanoma, melanoma with lymph node involvement or metastatic disease who have undergone complete resection, metastatic non-small cell lung cancer and progression on or after platinum-based chemotherapy, metastatic small cell lung cancer with progression after platinum-based chemotherapy and at least one other line of therapy, advanced renal cell carcinoma who have received prior antiangiogenic therapy, advanced renal cell carcinoma, classical Hodgkin lymphoma, recurrent or metastatic squamous cell carcinoma of the head and neck with disease progression on or after a platinum-based therapy, locally advanced or metastatic urothelial carcinoma, microsatellite instability-high (MSI-H) or mismatch repair deficient (dMMR) metastatic colorectal cancer, or hepatocellular carcinoma.

In some embodiments, the cancer is melanoma, non-small cell lung cancer, small cell lung cancer, head and neck squamous cell cancer, classical Hodgkin lymphoma, primary mediastinal large B-cell lymphoma, urothelial carcinoma, microsatellite instability-high cancer, gastric cancer, esophageal cancer, cervical cancer, hepatocellular carcinoma, merkel cell carcinoma, renal cell carcinoma, or endometrial carcinoma.

Other cancers include but are not limited to, uveal melanoma, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood and bone marrow cancers (e.g., advanced hematological malignancies, leukemia, e.g., acute myeloid leukemia (e.g., primary or secondary), acute lymphoblastic leukemia, acute lymphocytic leukemia, T cell leukemia, hematological malignancies, advanced myeloproliferative disorders, myelodysplastic syndrome, relapsed or refractory multiple myeloma, advanced myeloproliferative disorders), retinal, bladder, cervical, kidney, endometrial, meningioma, lymphoma, skin, uterine, lung, non small cell lung, nasopharyngeal carcinoma, neuroblastoma, solid tumor, hematologic malignancy, squamous cell carcinoma, testicular, thyroid, mesothelioma, brain vulval, sarcoma, intestine, oral, endocrine, salivary, spermatocyte seminoma, sporadic medullary thyroid carcinoma, non-proliferating testes cells, cancers related to malignant mast cells, non-Hodgkin's lymphoma, and diffuse large B cell lymphoma.

In some embodiments, the tumor is a solid tumor. In some embodiments, the solid tumor is locally advanced or metastatic, hi some embodiments, the solid tumor is refractory (e.g., resistant) after standard therapy.

Methods described herein can reduce, ameliorate or altogether eliminate the disorder, and/or its associated symptoms, to keep it from becoming worse, to slow the rate of progression, or to minimize the rate of recurrence of the disorder once it has been initially eliminated (i.e., to avoid a relapse). A suitable dose and therapeutic regimen may vary depending upon the specific compounds, combinations, and/or pharmaceutical compositions used and the mode of delivery of the compounds, combinations, and/or pharmaceutical compositions. In some embodiments, the method increases the average length of survival, increases the average length of progression-free survival, and/or reduces the rate of recurrence, of subjects treated with the combinations described herein in a statistically significant manner.

In some embodiments, the cancer is lung cancer (e.g., non-small cell lung cancer CNSCLC), e.g., KRAS mutant NSCLC; metastatic cancer), bone cancer, pancreatic cancer, skin cancer, cancer of the head or neck, cutaneous or intraocular melanoma, uterine cancer, ovarian cancer (e.g., unresectable low-grade ovarian, advanced or metastatic ovarian cancer), rectal cancer, cancer of the anal region, stomach cancer, colon cancer, breast cancer (e.g., triple-negative breast cancer (e.g., breast cancer which does not express the genes for the estrogen receptor, progesterone receptor, and Her2/neu)), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, mesothelioma (e.g., malignant pleural mesothelioma, e.g., surgical resectable malignant pleural mesothelioma) or a combination of one or more of the foregoing cancers. In some embodiments, the cancer is metastatic. In some embodiments, the abnormal cell growth is locally recurring (e.g. the subject has a locally recurrent disease, e.g., cancer).

### Additional Therapies

In some embodiments, the methods and compositions described herein is administered together with an additional therapy (e.g., cancer treatment). In one embodiment, a mixture of one or more compounds or pharmaceutical compositions may be administered with the combination described herein to a subject in need thereof. In yet another embodiment, one or more compounds or compositions (e.g., pharmaceutical compositions) may be administered with the combination described herein for the treatment or avoidance of various diseases, including, for example, cancer, diabetes, neurodegenerative diseases, cardiovascular disease, blood clotting, inflammation, flushing, obesity, aging, stress, etc. In various embodiments, combination therapies comprising a compound or pharmaceutical composition described herein may refer to (1) pharmaceutical compositions that comprise one or more compounds in combination with the combination described herein; and (2) co-administration of one or more compounds or pharmaceutical compositions described herein with the combination described herein, wherein the compound or pharmaceutical composition described herein have not been formulated in the same compositions. In some embodiments, the combinations described herein is administered with an additional treatment (e.g., an additional cancer treatment). In some embodiments, the additional treatment (e.g., an additional cancer treatment) can be administered simultaneously (e.g., at the same time), in the same or in separate compositions, or sequentially. Sequential administration refers to administration of one treatment before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days: 1, 2, 3, 4, 5, 6, 7, 8 or more weeks before) administration of an additional, e.g., secondary, treatment (e.g., a compound or therapy). The order of administration of the first and secondary compound or therapy can also be reversed.

Exemplary cancer treatments include, for example: chemotherapy, targeted therapies such as antibody therapies, immunotherapy, and hormonal therapy. Examples of each of these treatments are provided below.

### Chemotherapy

In some embodiments, a combination described herein is administered with a chemotherapy. Chemotherapy is the treatment of cancer with drugs that can destroy cancer cells. “Chemotherapy” usually refers to cytotoxic drugs which affect rapidly dividing cells in general, in contrast with targeted therapy. Chemotherapy drugs interfere with cell division in various possible ways, e.g., with the duplication of DNA or the separation of newly formed chromosomes. Most forms of chemotherapy target all rapidly dividing cells and are not specific for cancer cells, although some degree of specificity may come from the inability of many cancer cells to repair DNA damage, while normal cells generally can.

Examples of chemotherapeutic agents used in cancer therapy include, for example, antimetabolites (e.g., folic acid, purine, and pyrimidine derivatives) and alkylating agents (e.g., nitrogen mustards, nitrosoureas, platinum, alkyl sulfonates, hydrazines, triazenes, aziridines, spindle poison, cytotoxic agents, topoisomerase inhibitors and others). Exemplary agents include Aclarubicin, Actinomycin, Alitretinon, Altretamine, Aminopterin, Aminolevulinic acid, Amrubicin, Amsacrine, Anagrelide, Arsenic trioxide, Asparaginase, Atrasentan, Belotecan, Bexarotene, endamustine, Bleomycin, Bortezomib, Busulfan, Camptotnecin, Capecitabine, Carboplatin, Carboquone, Carmofur, Carmustine, Celecoxib, Chlorambucil, Chlormethine, Cisplatin, Cladribine, Clofarabine, Crisantaspase, Cyclophosphamide, Cytarabine, Dacarbazine, Dactinomycin, Daunorubicin, Decitabine, Demecolcine, Docetaxel, Doxorubicin, Efaproxiral, Elesclomol, Elsamitrucin, Enocitabine, Epirubicin, Estramustine, Etoglucid, Etoposide, Floxuridine, Fludarabine, Fluorouracil (5FU), Fotemustine, Gemcitabine, Gliadel implants, Hydroxycarbamide, Hydroxyurea, idarubicin, Ifosfamide, Irinotecan, Irofulven, Ixabepilone, Larotaxel, Leucovorin, Liposomal doxorubicin, Liposomal daunorubicin, Lonidamine, Lomustine, Lucanthone, Mannosulfan, Masoprocol, Melphalan, Mercaptopurine, Mesna, Methotrexate, Methyl aminolevulinate, Mitobronitol, Mitoguazone, Mitotane, Mitomycin, Mitoxantrone, Nedaplatin, Nimustine, Oblimersen, Omacetaxine, Ortataxel, Oxaliplatin, Paclitaxel, Pegaspargase, Pemetrexed, Pentostatin, Pirarubicin, Pixanlrone, Plicamycin, Porfimer sodium, Prednimustine, Procarbazine, Raltitrexed, Ranimustine, Rubitecan, Sapacitabine, Semustine, Sitimagene ceradenovec, Strataplatin, Streptozocin, Talaporfm, Tegafur-uracil, Temoporfin, Temozolomide, Teniposide, Tesetaxel, Testolactone, Tetranitrate, Thiotepa, Tiazofurine, Tioguanine, Tipifarnib, Topotecan, Trabectedin, Triaziquone, Triethylenemelamine, Triplatin, Tretinoin, Treosulfan, Trofosfamide, Uramustine, Valrubicin, Verteporfin, Vinblastine, Vincristine, Vindesine, Vinflunine, Vinorelbine, Vorinostat, Zorubicin, and other cytostatic or cytotoxic agents described herein.

Because some drugs work better together than alone, two or more drugs are often given at the same time or sequentially. Often, two or more chemotherapy agents are used as combination chemotherapy. In some embodiments, the chemotherapy agents (including combination chemotherapy) can be used in combination with a combination described herein.

### Targeted Therapy

In some embodiments, a combination described herein is administered with a targeted therapy. Targeted therapy constitutes the use of agents specific for the deregulated proteins of cancer cells. Small molecule targeted therapy drugs are generally inhibitors of enzymatic domains on mutated, overexpressed, or otherwise critical proteins within the cancer cell. Prominent examples are the tyrosine kinase inhibitors such as Axitinib, Bosutinib, Cediranib, desatinib, erolotinib, imatinib, gefitinib, lapatinib, Lestaurtinib, Nilotinib, Semaxanib, Sorafenib, Sunitinib, and Vandetanib, and also cyclin-dependent kinase inhibitors such as Alvocidib and Seliciclib. Monoclonal antibody therapy is another strategy in which the therapeutic agent is an antibody which specifically binds to a protein on the surface of the cancer cells. Examples include the anti-HER2/neu antibody trastuzumab (HERCEPTIN®) typically used in breast cancer, and the anti-CD20 antibody rituximab and Tositumomab typically used in a variety of B-cell malignancies. Other exemplary antibodies include Cetuximab, Panitumumab, Trastuzumab, Alemtuzumab, Bevacizumab, Edrecolomab, and Gemtuzumab. Exemplary fusion proteins include Aflibercept and Denileukin diftitox. In some embodiments, the targeted therapy can be used in combination with a combination described herein.

Targeted therapy can also involve small peptides as “homing devices” which can bind to cell surface receptors or affected extracellular matrix surrounding the tumor. Radionuclides which are attached to these peptides (e.g., RGDs) eventually kill the cancer cell if the nuclide decay s in the vicinity of the cell. An example of such therapy includes BEXXAR®.

### Immunotherapy

In some embodiments, a combination described herein is administered with an immunotherapy. Cancer immunotherapy refers to a diverse set of therapeutic strategies designed to induce the patient's own immune system to fight the tumor.

Contemporary methods for generating an immune response against tumors include intravesicular BCG immunotherapy for superficial bladder cancer, and use of interferons and other cytokines to induce an immune response in subjects with renal cell carcinoma and melanoma. Allogeneic hematopoietic stem cell transplantation can be considered a form of immunotherapy, since the donor's immune cells will often attack the tumor in a graft-versus-tumor effect. In some embodiments, the immunotherapy agents can be used in combination with a combination as described herein.

### Hormonal Therapy

In some embodiments, a combination described is administered with a hormonal therapy. The growth of some cancers can be inhibited by providing or blocking certain hormones. Common examples of hormone-sensitive tumors include certain types of breast and prostate cancers. Removing or blocking estrogen or testosterone is often an important additional treatment. In certain cancers, administration of hormone agonists, such as progestogens may be therapeutically beneficial. In some embodiments, the hormonal therapy agents can be used in combination with a combination described herein.

### Radiation Therapy

The combinations described herein can be used in combination with directed energy or particle, or radioisotope treatments, e.g., radiation therapies, e.g., radiation oncology, for the treatment of proliferative disease, e.g., cancer, e.g., cancer associated with cancer stem cells. The combinations described herein may be administered to a subject simultaneously or sequentially along with the directed energy or particle, or radioisotope treatments. For example, the combinations described herein may be administered before, during, or after the directed energy or particle, or radioisotope treatment, or a combination thereof. The directed energy or particle therapy may comprise total body irradiation, local body irradiation, or point irradiation. The directed energy or particle may originate from an accelerator, synchrotron, nuclear reaction, vacuum tube, laser, or from a radioisotope. The therapy may comprise external beam radiation therapy, teletherapy, brachy therapy, sealed source radiation therapy, systemic radioisotope therapy, or unsealed source radiotherapy. The therapy may comprise ingestion of, or placement in proximity to, a radioisotope, e.g., radioactive iodine, cobalt, cesium, potassium, bromine, fluorine, carbon. External beam radiation may comprise exposure to directed alpha particles, electrons (e.g., beta particles), protons, neutrons, positrons, or photons (e.g., radiowave, millimeter wave, microwave, infrared, visible, ultraviolet, X-ray, or gamma-ray photons). The radiation may be directed at any portion of the subject in need of treatment.

### Surgery

The combinations described herein can be used in combination with surgery, e.g., surgical exploration, intervention, biopsy, for the treatment of proliferative disease, e.g., cancer, e.g., cancer associated with cancer stem cells. The combinations described herein may be administered to a subject simultaneously or sequentially along with the surgery. For example, the combinations described herein may be administered before (preoperative), during, or after (post-operative) the surgery, or a combination thereof. The surgery may be a biopsy during which one or more cells are collected for further analysis. The biopsy may be accomplished, for example, with a scalpel, a needle, a catheter, an endoscope, a spatula, or scissors. The biopsy may be an excisional biopsy, an incisional biopsy, a core biopsy, or a needle biopsy, e.g., a needle aspiration biopsy. The surgery may involve the removal of localized tissues suspected to be or identified as being cancerous. For example, the procedure may involve the removal of a cancerous lesion, lump, polyp, or mole. The procedure may involve the removal of larger amounts of tissue, such as breast, bone, skin, fat, or muscle. The procedure may involve removal of part of, or the entirety of, an organ or node, for example, lung, throat, tongue, bladder, cervix, ovary, testicle, lymph node, liver, pancreas, brain, eye, kidney, gallbladder, stomach, colon, rectum, or intestine. In one embodiment, the cancer is breast cancer, e.g., triple negative breast cancer, and the surgery is a mastectomy or lumpectomy.

### Anti-Inflammatory Agents

A combination described herein can be administered with an anti-inflammatory agent. Anti-inflammatory agents can include, but are not limited to, non-steroidal anti-inflammatory agents (e.g., Salicylates (Aspirin (acetylsalicylic acid), Diflunisal, Salsalate), Propionic acid derivatives (Ibuprofen, Naproxen, Fenoprofen, Ketoprofen, Flurbiprofen, Oxaprozin, Loxoprofen), Acetic acid derivatives (Indomethacin, Sulindac, Etodolac, Ketorolac, Diclofenac, Nabumetone), Enolic acid (Oxicam) derivatives (Piroxicam, Meloxicam, Tenoxicam, Droxicam, Lomoxicam, Isoxicam), Fenamic acid derivatives (Fenamates) (Mefenamic acid, Meclofenamic acid, Flufenamic acid. Tolfenamic acid). Selective COX-2 inhibitors (Coxibs) (Ceiecoxib), Sulphonanilides (Nimesulide). Steriods (e.g. Hydrocortisone (Cortisol), Cortisone acetate, Prednisone, Prednisolone, Methylprednisolone, Dexamethasone, Betamethasone, Triamcinolone, Beclometasone, Fludrocortisone acetate, Deoxycorticosterone acetate, Aldosterone).

### Analgesic Agents

Analgesics can include but are not limited to, opiates (e.g. morphine, codeine, oxycodone, hydrocodone, dihydromorphine, pethidine, buprenorphine, tramadol, venlafaxine), paracetomal and Nonsteroidal anti-inflammatory agents (e.g., Salicylates (Aspirin (acetylsalicylic acid), Diflunisal, Salsalate), Propionic acid derivatives (Ibuprofen, Naproxen, Fenoprofen, Ketoprofen, Flurbiprofen, Oxaprozin, Loxoprofen), Acetic acid derivatives (Indomethacin, Sulindac, Etodolac, Ketorolac, Diclofenac, Nabumetone), Enolic acid (Oxicam) derivatives (Piroxicam, Meloxicam, Tenoxicam, Droxicam, Lomoxicam, Isoxicam), Fenamic acid derivatives (Fenamates) (Mefenamic acid, Meclofenamic acid, Flufenamic acid. Tolfenamic acid). Selective COX-2 inhibitors (Coxibs) (Ceiecoxib), Sulphonanilides (Nimesulide).

### Antiemetic Agents

A combination described herein can be administered with an antiemetic agent. Antiemetic agents can include, but are not limited to, 5-HT3 receptor antagonists (Dolasetron (Anzemet), Granisetron (Kytril, Sancuso), Ondansetron (Zofran), Tropisetron (Navoban), Palonosetron (Aloxi), Mirtazapine (Remeron)), Dopamine antagonists (Domperidone, Olanzapine, Droperidol, Haloperidol, Chlorpromazine, Promethazine, Prochlorperazine, Metoclopramide (Reglan), Alizapride, Prochlorperazine (Compazine, Stemzine, Buccastem, Stemetil, Phenotil), NK1 receptor antagonist (Aprepitant (Emend), Antihistamines (Cyclizine, Diphenhydramine (Benadryl), Dimenhydrinate (Gravol, Dramamine), Meclozine (Bonine, Antivert), Promethazine (Pentazine, Phenergan, Promacot), Hydroxyzine), benzodiazapines (Lorazepam, Midazolam), Anticholinergics (hyoscine), steroids (Dexamethasone).

### Combinations

The phrase, “in combination with,” and the terms “co-administration,” “co-administering,” or “co-providing”, as used herein in the context of the administration of a compound described herein or a therapy described herein, means that two (or more) different compounds or therapies are delivered to the subject during the course of the subject's affliction with the disease or disorder (e.g., a disease or disorder as described herein, e.g., cancer), e.g., two (or more) different compounds or therapies are delivered to the subject after the subject has been diagnosed with the disease or disorder (e.g., a disease or disorder as described herein, e.g., cancer) and before the disease or disorder has been cured or eliminated or treatment has ceased for other reasons.

In some embodiments, the delivery of one compound or therapy is still occurring when the delivery of the second begins, so that there is overlap in terms of administration. This is sometimes referred to herein as “simultaneous” or “concurrent delivery.” In other embodiments, the delivery of one compound or therapy ends before the delivery of the other compound or therapy begins. In some embodiments of either case, the treatment (e.g., administration of compound, composition, or therapy) is more effective because of combined administration. For example, the second compound or therapy is more effective, e.g., an equivalent effect is seen with less of the second compound or therapy, or the second compound or therapy reduces symptoms to a greater extent, than would be seen if the second compound or therapy were administered in the absence of the first compound or therapy, or the analogous situation is seen with the first compound or therapy. In some embodiments, delivery is such that the reduction in a symptom, or other parameter related to the disorder is greater than what would be observed with one compound or therapy delivered in the absence of the other. The effect of the two compounds or therapies can be partially additive, wholly additive, or great than additive (e.g., synergistic). The delivery can be such that the first compound or therapy delivered is still detectable when the second is delivered.

In some embodiments, the first compound or therapy and second compound or therapy can be administered simultaneously (e.g., at the same time), in the same or in separate compositions, or sequentially. Sequential administration refers to administration of one compound or therapy before (e.g., immediately before, less than 5, 10, 15, 30, 45, 60 minutes; 1, 2, 3, 4, 6, 8, 10, 12, 16, 20, 24, 48, 72, 96 or more hours; 4, 5, 6, 7, 8, 9 or more days; 1, 2, 3, 4, 5, 6, 7, 8 or more weeks before) administration of an additional, e.g., secondary, compound or therapy. The order of administration of the first and secondary compound or therapy can also be reversed.

The combinations described herein can be a first line treatment for abnormal cell growth, e.g., cancer, i.e., it is used in a patient who has not been previously administered another drug intended to treat the cancer; a second line treatment for the cancer, i.e., it is used in a subject in need thereof who has been previously administered another drug intended to treat the cancer; a third or fourth treatment for the cancer, i.e., it is used in a subject who has been previously administered two or three other drugs intended to treat the cancer.

### Administration and Dosage

The combinations of this invention may be administered orally, parenterally, topically, rectally, or via an implanted reservoir, preferably by oral administration or administration by injection. In some cases, the pH of the composition (e.g., pharmaceutical composition) may be adjusted with pharmaceutically acceptable acids, bases or buffers to enhance the stability or efficacy of the composition.

In some embodiments, the subject is administered the composition (e.g., pharmaceutical composition) orally. In some embodiments the composition (e.g., pharmaceutical composition) is be orally administered in any orally acceptable dosage form including, but not limited to, liqui-gel tablets or capsules, syrups, emulsions and aqueous suspensions. Liqui-gels may include gelatins, plasticisers, and/or opacifiers, as needed to achieve a suitable consistency and may be coated with enteric coatings that are approved for use, e.g., shellacs. Additional thickening agents, for example gums, e.g., xanthum gum, starches, e.g., corn starch, or glutens may be added to achieve a desired consistency of the composition (e.g., pharmaceutical composition) when used as an oral dosage. If desired, certain sweetening and/or flavoring and/or coloring agents may be added.

In some embodiments, the subject is administered the composition (e.g., pharmaceutical composition) in a form suitable for oral administration such as a tablet, capsule, pill, powder, sustained release formulations, solution, and suspension. The composition (e.g., pharmaceutical composition) may be in unit dosage forms suitable for single administration of precise dosages. Pharmaceutical compositions may comprise, in addition to a compound as described herein a pharmaceutically acceptable carrier, and may optionally further comprise one or more pharmaceutically acceptable excipients, such as, for example, stabilizers, diluents, binders, and lubricants. In addition, the tablet may include other medicinal or pharmaceutical agents, carriers, and or adjuvants. Exemplary pharmaceutical compositions include compressed tablets (e.g., directly compressed tablets).

Tablets are also provided comprising the active or therapeutic ingredient (e.g., compound as described herein). In addition to the active or therapeutic ingredients, tablets may contain a number of inert materials such as carriers. Pharmaceutically acceptable carriers can be sterile liquids, such as water and oils, including those of petroleum, animal, vegetable or synthetic origin, such as peanut oil, sesame oil and the like. Saline solutions and aqueous dextrose can also be employed as liquid earners. Oral dosage forms for use in accordance with the present invention thus may be formulated in conventional manner using one or more pharmaceutically acceptable carriers comprising excipients and auxiliaries, which facilitate processing of the active ingredients into preparations which, can be used pharmaceutically. Excipients can impart good powder flow and compression characteristics to the material being compressed. Examples of excipients are described, for example, in the Handbook of Pharmaceutical Excipients (5^{th} edition), Edited by Raymond C Rowe, Paul J. Sheskey, and Sian C. Owen; Publisher: Pharmaceutical Press.

For oral administration, the active ingredients, e.g., the compound as described herein can be formulated readily by combining the active ingredients with pharmaceutically acceptable carriers well known in the art. Such carriers enable the active ingredients of the invention to be formulated as tablets, pills, capsules, liquids, gels, syrups, slurries, powders or granules, suspensions or solutions in water or non-aqueous media, and the like, for oral ingestion by a subject. Pharmacological preparations for oral use can be made using a solid excipient, optionally grinding the resulting mixture, and processing the mixture of granules, after adding suitable auxiliaries if desired, to obtain, for example, tablets. Suitable excipients such as diluents, binders or disintegrants may be desirable.

The dosage may vary depending upon the dosage form employed and the route of administration utilized. The exact formulation, route of administration and dosage can be chosen by the individual physician in view of the patient's condition. (See e.g., Fingl, et al., 1975, in 'he Pharmacological Basis of Therapeutics”). Lower or higher doses than those recited above may be required. Specific dosage and treatment regimens for any particular subject will depend upon a variety of factors, including the activity of the specific compound employed, the age, body weight, general health status, sex, diet, time of administration, rate of excretion, drug combination, the severity and course of the disease, condition or symptoms, the subject's disposition to the disease, condition or symptoms, and the judgment of the treating physician. A course of therapy can comprise one or more separate administrations of a compound as described herein. A course of therapy can comprise one or more cycles of a compound as described herein.

In some embodiments, a cycle, as used herein in the context of a cycle of administration of a drug, refers to a period of time for which a drug is administered to a patient. For example, if a drug is administered for a cycle of 21 days, the periodic administration, e.g., daily or twice daily, is given for 21 days. A drug can be administered for more than one cycle. Rest periods may be interposed between cycles. A rest cycle may be 1, 2, 4, 6, 8, 10, 12, 16, 20, 24 hours, 1, 2, 3, 4, 5, 6, 7 days, or 1, 2, 3, 4 or more weeks in length.

Oral dosage forms may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

### EXAMPLES

In order that the invention described herein may be more fully understood, the following examples are set forth. The examples described in this application are offered to illustrate the pharmaceutical compositions and methods provided herein and are not to be construed in any way as limiting their scope.

### Example 1. Combination Therapy of VS-6766 and Anti-PD-1 Antibody

### Materials and Methods

RNA Extraction and qRT-PCR

Cells were seeded into 6-cm dishes and after an incubate overnight (17-22 hrs) cells were treated with VS-6766 for 48 hours at a concentration that gave an 80% cell viability (1 μM for A549, TOV21G and WM115, and 300 nM for SKMEL5 and IGR-1). RNA was extracted using RNeasy Plus Mini kit (Qiagen) and converted to cDNA using the cDNA by High-Capacity cDNA Reverse Transcription Kit (Applied Biosystems). TaqMan qPCR was run using HLA-A, B2M, TAP1 and TAP-2 probes from Applied Biosystems. VIC-GAPDH probe as the house keeping gene. Each PCR reaction will be run in triplicate wells. Expression levels were computed as the difference (ΔCT) between the target gene CT and GAPDH CT.

### Syngeneic Tumor Mouse Studies

CT26 tumor cells were obtained from ATCC and Balb/c mice were obtained from Shanghai Lingchang Biotechnology. Tumor challenge was initiated by subcutaneous inoculation of 3×105 CT26 tumor cell suspensions into the right flank of recipient mice. Tumor sizes (mm3) were measured and calculated as described above. Once tumors reached an average volume of 50-80 mm3, mice were sorted into 4 groups (n=10): vehicle (5% DMSO, 10% HPCD in sterile water, oral dosing (PO), once per day (QD), 28 days)+isotype control (60 mg/mouse intraperitoneally (IP) biweekly, 4 doses; rat IgG2a clone 2A3, BioXcell), VS-6766 (0.5 mg/kg PO QD, 28 days)+isotype control, anti-PD-1 (60 μg/mouse IP biweekly, 4 doses; clone RMP1-14, BioXCell), and VS-6766+anti-PD-1. Tumors and body weights were measured 3 times per week for the duration of the study. At the time of routine monitoring, the animals were checked for any effects of tumor growth and treatments on normal behavior such as mobility, food and water consumption (by looking only), and body weight gain/loss, eye/hair matting and any other abnormal effect. For Kaplan-Meier analysis, an event was defined by death or tumor growth beyond 2,000 mm³.

In another study, once tumors reached an average volume of 50-80 mm3, mice were sorted into 7 groups (n=10): vehicle 1 (5% DMSO, 10% HPCD in sterile water, PO QD, 28 days)+vehicle 2 (0.5% CMC, 0.1% Tween 80 in sterile water, PO BID, 28 days)+isotype control (60 mg/mouse IP biweekly, 4 doses; rat IgG2a clone 2A3, BioXcell); VS-6766 (0.5 mg/kg PO QD, 28 days)+vehicle 2+isotype control; vehicle 1+vehicle 2+anti-PD-1 (60 μg/mouse IP biweekly, 4 doses; clone RMP1-14, BioXCell); vehicle 1+VS-4718 (50 mg/kg PO BID, 28 days)+isotype control; VS-6766+vehicle 2+anti-PD-1; vehicle 1+VS-4718+anti-PD-1; and VS-6766+VS-4718+anti-PD-1.

### Immune Memory Studies

Tumor-free mice were injected with 6×105 CT26 tumor cells in the contralateral flank with no further treatment. Naïve mice were used as positive control for tumor cell inoculation. Tumor sizes (mm3) were measured and calculated as described above. Tumors and body weights were measured 3 times per week for the duration of the study.

Thirty days after tumor re-challenge, blood and spleens from mice previously treated with duvelisib+anti-PD-1 were collected and the percentage of memory CD4+ and CD8+ T cells in blood and spleen were measured. Naïve mice were used as control.

### Results

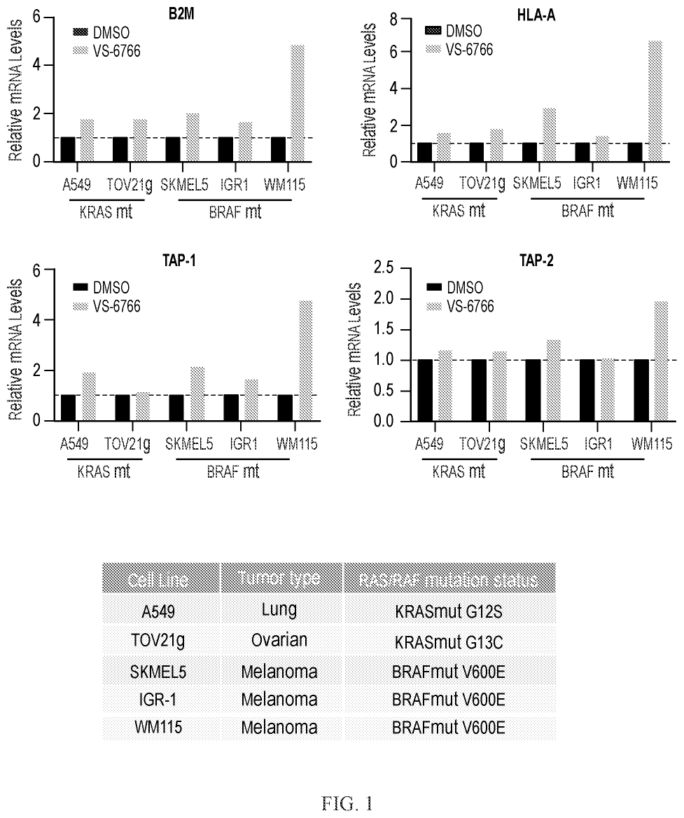

In nonclinical studies VS-6766 treatment has been shown to increase tumor antigen presentation via upregulation of major histocompatibility complex class I (MHC-I) expression, including beta 2-microglobulin (B2M), human leukocyte antigens A (HLA-A), transporter associated with antigen processing 1 (TAP-1) and transporter associated with antigen processing 2 (TAP-2), in multiple KRAS/BRAF mutant human cancer cell lines (FIG. 1). RAF/MEK inhibition by VS-6766 for 48 h led to increased HLA-A, along with TAP1, TAP2 and β2M in in KRAS/BRAF mutant human cancer cell lines. The MHC-I presents antigenic peptides to tumor-specific CD8+ T cells and is essential for CD8+ cytotoxic T-cell responses. Thus, reduced cell-surface presentation of tumor antigens on MHC-I is an important obstacle to effective immunotherapy.

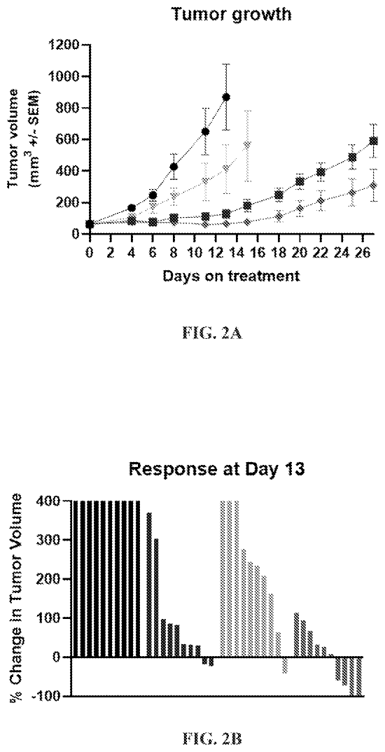

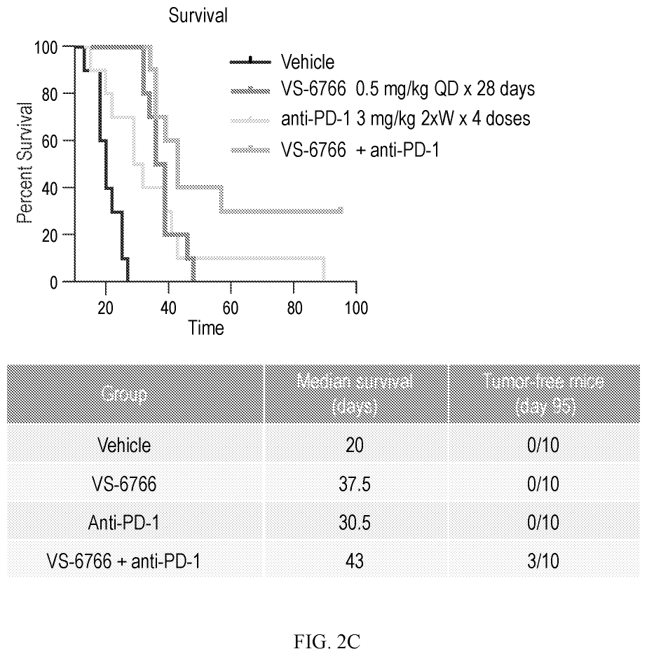

These effects on different components of the MHC-I complex make VS-6766 a good candidate for combination therapy with immuno-oncology agents, including checkpoint inhibitors (anti-PD-1 and anti-PD-L1). Indeed, in the CT26 KRAS(G12D)-mutant colorectal cancer mouse models, it was shown that although VS-6766 and the PD-1 antibody (anti-PD-1) can each delay tumor growth, combination of VS-6766 and the anti-PD-1 antibody resulted in increased antitumor efficacy and prolonged survival. FIG. 2A shows the tumor volume changes in CT26 xenografts after treatment with VS-6766 (0.5 mg/kg QD×28 days), anti-PD-1 antibody (3 mg/kg BIW×4 doses), VS-6766+anti-PD-1 or vehicle. FIG. 2B shows the change in tumor volumes in CT26 mice treated with VS-6766, anti-PD-1 antibody, VS-6766+anti-PD-1 or vehicle for 13 days. FIG. 2C shows the Kaplan-Meier survival curve of CT26 mice treated with VS-6766, anti-PD-1 antibody, VS-6766+anti-PD-1 or vehicle.

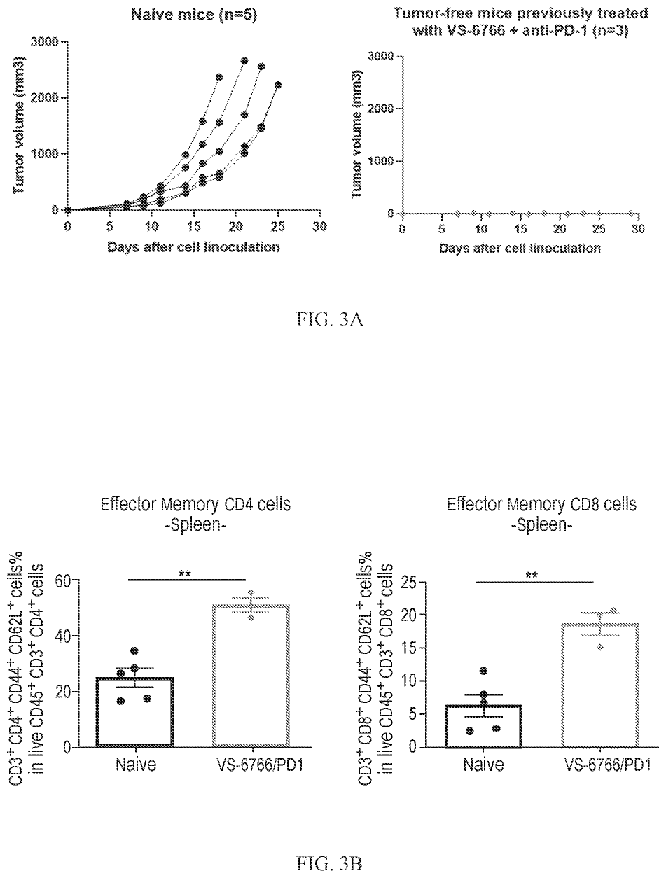

To determine the ability of VS-6766 to induce a durable antitumor T cell response, CT26 tumor-bearing mice that were tumor-free after previous treatment with the combination of VS-6766+anti-PD-1 (n=3) were subsequently re-challenged with CT26 cells in the absence of further treatment. All tumor-free mice previously treated with VS-6766+anti-PD-1 were able to reject a re-challenge with CT26 tumor cells for a period of 25 days (FIG. 3A). Additionally, increased numbers of CD8 memory T cells (CD8+CD44+CD62L−) and CD4 memory T cells (CD4+CD44+CD62L−) (FIG. 3B) were observed in the spleen of tumor-free mice previously treated with VS-6766+anti-PD-1 compared to untreated naïve control mice. (FIG. 3B shows percentage of memory CD4+ and CD8+ T cells in spleen for untreated naïve mice and mice previously treated with the VS-6766+anti-PD-1 combination treatment.) These results indicate that combination of VS-6766+anti-PD-1 induces a specific and durable antitumor immunologic memory response in tumor-free mice.

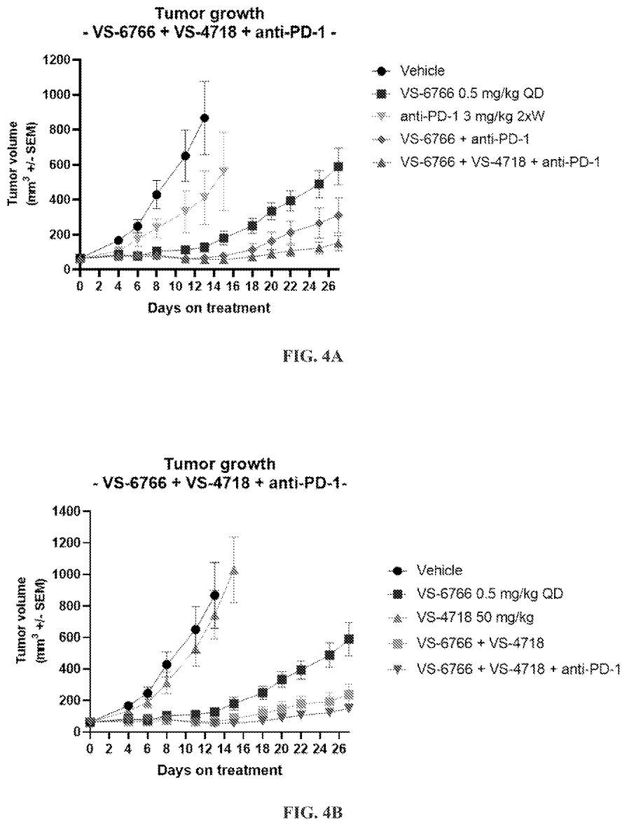

Mice bearing CT26 colorectal tumors were randomized once tumors reached 50-80 mm3 and treated with VS-6766, VS-4718, VS-6766+VS-4718, anti-PD-1, VS-6766+anti-PD-1 or VS-6766+VS-4718+anti-PD-1. Tumor sizes (mm³) were measured 3 times per week for the duration of the study. The antitumor effect of VS-6766+anti-PD-1 in combination with a FAK inhibitor (FAKi; VS-4718) was assessed in the CT26 KRAS(G12D)-mutant colorectal cancer mouse models (FIGS. 4A and 4B). It was shown that addition of FAKi enhances the antitumor efficacy of the VS-6766/anti-PD-1 combination.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of treating a cancer in a subject in need thereof, the method comprising administering to the subject a dual RAF/MEK inhibitor or a pharmaceutically acceptable salt thereof in combination with an anti-PD-1 antibody, thereby treating the subject.

2. The method of claim 1, wherein the dual RAF/MEK inhibitor is VS-6766 or a pharmaceutically acceptable salt thereof.

3. The method of claim 1 or 2, wherein the dual RAF/MEK inhibitor is dosed at least once a week.

4. The method of any one of claims 1-3, wherein the dual RAF/MEK inhibitor is dosed twice a week.

5. The method of claim 1 or 2, wherein the dual RAF/MEK inhibitor is dosed twice a week cyclically for three weeks on and then one week off.

6. The method of claim 5, wherein the cycle is repeated at least once.

7. The method of any one of claims 1-6, wherein the dual RAF/MEK inhibitor is dosed at about 0.8 mg to about 10 mg per administration.

8. The method of any one of claims 1-7, wherein the dual RAF/MEK inhibitor is dosed at about 3.2 mg per administration.

9. The method of any one of claims 1-7, wherein the dual RAF/MEK inhibitor is dosed at about 4 mg per administration.

10. The method of any one of claims 1-9, wherein the anti-PD-1 antibody is selected from the group consisting of balstilimab, camrelizumab, cemiplimab, dostarlimab, geptanolimab, nivolumab, pembrolizumab, penpulimab, pidilizumab, prolgolimab, retifanlimab, sasanlimab, serplulimab, serplulimab, sintilimab, spartalizumab, sulituzumab, tebotelimab, teripalimab, tislelizumab, toripalimab, toripalimab, zimberelimab, AMP-224, AMP-514, AT-16201, AVI-102, BAT-1308, BH-2950, BSI-050K01, CB-201, CYTO-101, DB-004, EX-105, EX-108, GNR-051, HAB-21, IBI-319, IBI-321, IKT-202, IMU-201, JS-201, LBL-006, LBL-024, LD-01, LQ-005, LQ-008, MD-402, OT-2, PE-0105, PF-07209960, PH-762, REGN-PD-1/XX, R07121661, SAUG-1, SCT-I10A, SG-001, SG001, SI-B003, SL-279137, SSI-361, STI-A1110, STM-418, Sym-021, TSR-075, TY101, Twist-PD-1, XmAb-TGFβR2, XmAb-YYCD28, XmAb20717, XmAb23104, YBL-006, YBL-019, and mDX-400.

11. The method of any one of claims 1-10, wherein the anti-PD-1 antibody is dosed at least once a week.

12. The method of any one of claims 1-10, wherein the anti-PD-1 antibody is dosed every two weeks.

13. The method of any one of claims 1-10, wherein the anti-PD-1 antibody is dosed every three weeks.

14. The method of any one of claims 1-10, wherein the anti-PD-1 antibody is dosed every four weeks.

15. The method of any one of claims 1-10, wherein the anti-PD-1 antibody is dosed every six weeks.

16. The method of any one of claims 1-15, wherein the anti-PD-1 antibody is dosed at about 100 mg to about 1000 mg per administration.

17. The method of any one of claims 1-16, wherein the anti-PD-1 antibody is administered parenterally.

18. A method of treating a cancer in a subject in need thereof, the method comprising administering to the subject a dual RAF/MEK inhibitor or a pharmaceutically acceptable salt thereof in combination with an anti-PD-L1 antibody, thereby treating the subject.

19. The method of claim 18, wherein the dual RAF/MEK inhibitor is VS-6766 or a pharmaceutically acceptable salt thereof.

20. The method of claim 18 or 19, wherein the anti-PD-L1 antibody is selected from the group consisting of atezolizumab, avelumab, durvalumab, envafolimab, socazolimab, sugemalimab, ABM-101, AP-505, APL-801, ATG-101, AVA-027, AUNP12, B-1961, BH-3120, BMS-986189, BPI-9220, BPI-9320, CA-170, CCX-559, CK-301, CS-17938, CTX-8371, CYTCDR-2, DB-003, DPDL-1E, DR-30207, DSP-105, DSP-502, EI-011, EI-014, EMB-08, ENN-101, ENN-102, GB-7003, Gensci-047, HB-0025, HB-0028, HB-0036, HBM-7015, IBI-327, IGM-7354, IKT-201, IMC-2101, IMC-2102, IMGS-002, IMM-2510, INBRX-105, JBI-426, JNB-809, JNB-809, JNB-813, JNB-813, KN-052, KN035, KY-1043, LP-008, LQ-002, LQ-004, LVGN-1673, LY-3434172, LYN-102, MCLA-145, MEDI-7526, PH-790, PM-1003, PRS-344, Q-1802, QL-301, QLS31901, RC98, SHR-1316, SHR-1701, SIM-236, SL-279252, SL-279258, SLSP-03, SNA-02, STT-01, TI-1007, TJ-L1C4, TJ-L1D5, TJ-L1H3, TJ-L117, TJL-14B, TS1905, TST-005, TTXsiPDL-1, TXB-4BC3, VXM-10, YBL-007, YBL-008, YBL-009, YBL-013, YBL-016, and YBL-020.

21. The method of any one of claims 18-20, wherein the anti-PD-L1 antibody is administered parenterally.

22. The method of any one of claims 18-21, wherein the anti-PD-L1 antibody is administered every two weeks.

23. The method of any one of claims 18-21, wherein the anti-PD-L1 antibody is administered every three weeks.

24. The method of any one of claims 18-21, wherein the anti-PD-L1 antibody is administered every four weeks.

25. The method of any one of claims 18-24, wherein the anti-PD-L1 antibody is dosed at about 100 mg to about 2000 mg per administration.

26. The method of any one of claims 18-25, wherein the dual RAF/MEK inhibitor is dosed at least once a week.

27. The method of any one of claims 18-26, wherein the dual RAF/MEK inhibitor is dosed twice a week.

28. The method of any one of claims 18-25, wherein the dual RAF/MEK inhibitor is dosed twice a week cyclically for three weeks on and then one week off.

29. The method of claim 28, wherein the cycle is repeated at least once.

30. The method of any one of claims 18-29, wherein the dual RAF/MEK inhibitor is dosed at about 0.8 mg to about 10 mg per administration.

31. The method of any one of claims 18-30, wherein the dual RAF/MEK inhibitor is dosed at about 3.2 mg per administration.

32. The method of any one of claims 18-30, wherein the dual RAF/MEK inhibitor is dosed at about 4 mg per administration.

33. The method of any one of claims 1-32, wherein the method further comprises administering to the subject a FAK inhibitor or a pharmaceutically acceptable salt thereof.

34. The method of claim 33, wherein the FAK inhibitor is defactinib.

35. The method of claim 33 or 34, wherein the FAK inhibitor is dosed twice daily.

36. The method of claim 33 or 34, wherein the FAK inhibitor is dosed once daily.

37. The method of any one of claims 33-36, wherein the FAK inhibitor is dosed at about 100 mg to about 1000 mg per administration.

38. The method of any one of claims 33-37, wherein the FAK inhibitor is dosed at about 200 mg to about 400 mg per administration.

39. The method of any one of claims 33-38, wherein the FAK inhibitor is dosed at about 200 mg per administration.

40. The method of any one of claims 33-38, wherein the FAK inhibitor is dosed at about 400 mg per administration.

41. The method of any one of claims 33-40, wherein the FAK inhibitor is administered orally.

42. A method of treating a cancer in a subject in need thereof, the method comprising administering to the subject a dual RAF/MEK inhibitor or a pharmaceutically acceptable salt thereof in combination with an anti-PD-1 antibody and a FAK inhibitor, thereby treating the subject.

43. A method of treating a cancer in a subject in need thereof, the method comprising administering to the subject a dual RAF/MEK inhibitor or a pharmaceutically acceptable salt thereof in combination with an anti-PD-L1 antibody and a FAK inhibitor, thereby treating the subject.

44. The method of claim 42 or 43, wherein the dual RAF/MEK inhibitor is VS-6766 or a pharmaceutically acceptable salt thereof.

45. The method of any one of claims 1-44, wherein the cancer is a cancer characterized as having a RAS mutation.

46. The method of any one of claims 1-45, wherein the cancer is a cancer characterized as having a RAF mutation.

47. The method of any one of claims 1-44, wherein the cancer is a cancer characterized as having a KRAS, NRAS, HRAS, and/or BRAF mutation.

48. The method of any one of claims 1-47, wherein the cancer is lung adenocarcinoma, colorectal cancer, uveal melanoma, ovarian cancer, uterine endometrioid carcinoma, bladder urothelial carcinoma, breast invasive lobular carcinoma, cervical squamous cell carcinoma, cutaneous melanoma, endocervical adenocarcinoma, hepatocellular carcinoma, pancreatic adenocarcinoma, biphasic type pleural mesothelioma, renal clear cell carcinoma, renal clear cell carcinoma, stomach adenocarcinoma, tubular stomach adenocarcinoma, uterine carcinosarcoma, or uterine malignant mixed Mullerian tumor.
